(19) United States
(12) Patent Application Publication
(10) Pub. No.: US 2024/0052336 A1
(43) Pub. Date: Feb. 15, 2024

(54) Filter-based extracellular vesicle nucleic acid isolation method

(71) Applicants: SHOWA DENKO MATERIALS (AMERICA), INC., San Jose, CA (US); HITACHI CHEMICAL CO., LTD., Tokyo (JP)

(72) Inventor: Taku Murakami, San Jose, CA (US)

(73) Assignees: SHOWA DENKO MATERIALS (AMERICA), INC., San Jose, CA (US); SHOWA DENKO MATERIALS CO., LTD., Tokyo, CA (JP)

(21) Appl. No.: 17/766,185

(22) PCT Filed: Sep. 30, 2020
(86) PCT No.: PCT/US2020/053526

§ 371 Date: Apr. 1, 2022

Related U.S. Application Data
(60) Provisional application No. 62/908,658, filed on Oct. 1, 2019.

(51) Int. Cl. C12N 15/10

(52) U.S. Cl. CPC C12N 15/1017

(57) ABSTRACT

The present disclosure relates to systems, devices and methods for the enhanced efficiency of capturing agents of interest from a sample.

### BACKGROUND

The present disclosure relates to systems, devices and methods for the enhanced efficiency of capturing agents of interest from a sample.

The sample may be a biological fluid sample in some embodiments, while in other embodiments, non-biological samples are used. For example, in several embodiments, environmental water samples are passed through the devices as disclosed herein in order to assess, for example, mineral content, pollution levels, chemical or toxin content, presence of pathogens, etc.

### DESCRIPTION OF RELATED ART

Often it is desirable to extract certain components from a sample. For example, many medical tests analyze biomarkers in a sample, including a fluid sample (e.g., blood, urine, etc.) taken from a patient. Diagnosis or prognosis may be derived from identification of a biomarker or a biochemical pattern that is not present in healthy patients or is altered from a previously obtained patient sample.

Frequently, the use of bodily fluids to isolate or detect a biomarker significantly dilutes the biomarker. Moreover, most biomarkers are produced in low or even moderate amounts in tissues and bodily fluids. Diagnosis or prognosis is likely less accurate when the compounds of interest are present at low concentrations.

Given that accurate diagnosis may be hampered (or even impossible) when a target compound of interest is present in a biological sample at low concentrations, there is a need for devices and methods for extracting biomarkers and other components of interest from a sample of a patient without unduly lowering the concentration of the target biomarker. Extraction of the components of interest is beneficial in many contexts including, but not limited to, filtration, purification, isolation, and enrichment.

Thus, several embodiments of the devices and methods allow extraction of target components from liquids. In particular, the devices and methods disclosed herein are useful for capturing from biological fluids nucleic acids, exosomes, vesicles, and other circulating membrane bound nucleic acid and/or protein-containing structures. However, as the devices and methods disclosed herein permit extraction of organic and non-organic compounds, the devices and methods disclosed herein are applicable to fluid samples of biological or non-biological origin.

Conventional methods of vesicle isolation often involve ultracentrifugation in order to separate the vesicles from other matter in a biological sample. Ultracentrifugation is accomplished through the use of expensive and potentially hazardous equipment. Moreover, ultracentrifugation often results in samples being collected in multiple tubes. Consequently, ultracentrifugation is sometimes an impractical or impossible technique for many laboratories/clinical sites. After vesicles are isolated from biological fluids, biomarkers encapsulated in or associated with the vesicles such as RNA, DNA, protein, etc. are isolated through lysing the vesicles and purifying the biomarker by conventional isolation methods of biomolecules such as organic solvent extraction. As those methods require toxic organic solvents such as phenol and chloroform and labor-intensive/time-consuming protocols, the conventional methods of vesicle biomarker isolation using organic solvent extraction are not practical, either.

Therefore, in one aspect, provided herein are devices and methods for capture of exosomes, vesicles, and other circulating membrane-bound nucleic acid and/or protein-containing structures that are released from cells into biological fluids, and isolation of the biological biomarkers associated with these vesicles using the same devices. In several embodiments the devices and methods as disclosed herein provide several advantages over traditional techniques for isolation of vesicle associated biomarkers, such as ultracentrifugation and organic solvent extraction. For example, in some embodiments, the devices and methods disclosed herein allows efficient isolation of vesicle associated biomarkers existing at low concentrations in a large volume of samples. By applying multiple sample aliquots to a device, vesicles are concentrated on the devices. In some embodiments, vesicle yield is increased by re-passing the filtrate of a sample aliquot through the device. The vesicles captured in the device are lysed in the device and the vesicle associated biomarkers are isolated using the same devise without diluting the low concentration of biomarkers in lysis solutions. After removing the other matter from the samples, pure vesicle associated biomarkers are released in a small volume of elution solution.

### BRIEF DESCRIPTION OF FIGURES

FIG. 1 depicts a schematic protocol of exemplary extracellular vesicle (EV) RNA isolation. The first step is to add biological samples containing vesicles to a capture device (Step 1). The vesicles are captured by a filter material through size exclusion/electrostatic interactions by filtering the samples through the filter material by centrifugation (Step 2). Following the vesicle capture, adding Lysis/Binding solution to the vesicle-captured filter lyses the vesicles and releases vesicle associated RNA (Step 3). Since the Lysis/Binding solution is configured to allow binding between RNA and the filter material, released RNA from the vesicle are captured by the filter material immediately after the vesicle lysis. Remained Lysis/Binding solution is removed by filtration after vesicle lysis and RNA capture (Step 4). Washing the filter material by Wash solution removes contaminants that may inhibit downstream enzymatic/non-enzymatic applications (Steps 5, 6). Wash step can be repeated several times to obtain the desired purity of RNA. Elution of RNA from the filter can be done by adding Elution solution (Steps 7, 8).

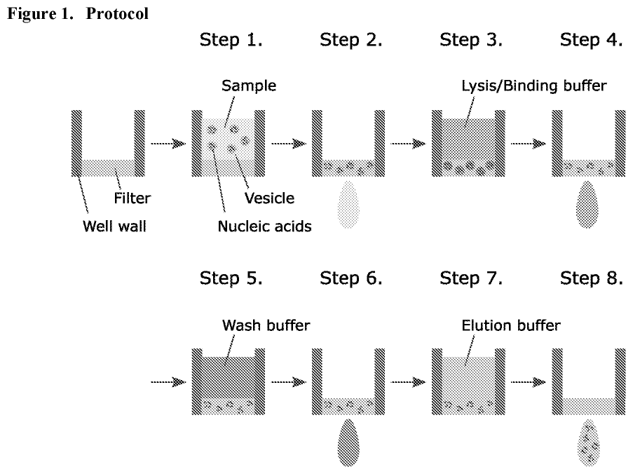

FIG. 2 depicts results from comparing exemplary filter materials.

FIG. 3 depicts results from comparing additional exemplary filter materials.

FIG. 4 depicts comparing lysis/binding solutions for urine EV mRNA extraction.

FIG. 5 depicts comparing lysis/binding solutions for plasma EV mRNA extraction.

FIG. 6 depicts comparing lysis/binding solutions for urine EV mRNA extraction.

FIG. 7 depicts comparing lysis/binding solutions for urine EV miRNA extraction.

FIG. 8 depicts comparing lysis/binding solutions for plasma EV miRNA extraction.

FIG. 9 depicts comparing lysis/binding solutions for urine EV mRNA extraction.

FIG. 10 depicts comparing wash buffers for urine EV mRNA extraction.

FIG. 11 depicts comparing elution for urine EV mRNA extraction.

FIG. 12 depicts comparing gene expression profile methods for urine EV mRNA extraction.

FIG. 13 depicts comparing gene expression profile methods for urine EV miRNA extraction.

FIG. 14 depicts EV RNA profile analysis results.

### DESCRIPTIONS

Due to the rapid rate of nucleic acid degradation in the extracellular environment, conventional understanding suggests that many tissues are unable to provide nucleic acid that would be suitable as a diagnostic target because the nucleic acids would be degraded before they could be used as a template for detection. However, extracellular RNA (as well as other biomarkers disclosed herein) is often associated with one or more different types of membrane particles (ranging in size of 50-80 nm), exosomes (ranging in size of 50-100 nm), exosome-like vesicles (ranging in size of 20-50 nm), and microvesicles (ranging in size of 100-1000 nm). Other vesicle types may also be captured, including, but not limited to, nanovesicles, vesicles, dexosomes, blebs, prostasomes, microparticles, intralumenal vesicles, endosomal-like vesicles or exocytosed vehicles. As used herein, the terms “exosomes”, “vesicles” and “extracellular vesicles (EV)” are used in accordance with their respective ordinary meanings in this field and shall also be read to include any shed membrane bound particle that is derived from either the plasma membrane or an internal membrane. For clarity, the terms describing various types of vesicles shall, unless expressly stated otherwise, be generally referred to as vesicles or exosome. Exosomes can also include cell-derived structures bounded by a lipid bilayer membrane arising from both herniated evagination (e.g., blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen, including but not limited to tumor-derived microRNAs or intracellular proteins. Exosomes can also include membrane fragments. Circulating tumor-derived exosomes (CTEs) as referenced herein are exosomes that are shed into circulation or bodily fluids from tumor cells. CTEs, as with cell-of-origin specific exosomes, typically have unique biomarkers that permit their isolation from bodily fluids in a highly specific manner. In some embodiments, the diameter of the vesicle described herein may be about 10, 15, 20, 25, 30, 35, 40, 45, 50, 55, 60, 65, 70, 75, 80, 85, 90, 95, 100, 105, 110, 115, 120, 130, 140, 150, 160, 170, 180, 190, 200, 300, 400, 500, 600, 700, 800, 900 nm or more. In some embodiments, the diameter of the vesicle described herein may be about 10, 15, 20, 25, 30, 35, 40, 45, 50, 55, 60, 65, 70, 75, 80, 85, 90, 95, 100, 105, 110, 115, 120, 130, 140, 150, 160, 170, 180, 190, 200, 300, 400, 500, 600, 700, 800, 900, 1000, 1100, 1200 nm or less. As used herein with respect to any numerical value, the term “about” means the value indicated plus or minus 30, 20, 10 or 5%.

In one aspect, the present disclosure is related to a method of isolating nucleic acids from vesicles in a sample. As achieved by several embodiments disclosed herein, selective isolation of any of such type of vesicles allows for isolation and analysis of their associated nucleic acids including RNA (such as messenger RNA (mRNA), microRNA (miRNA), ribosomal RNA (rRNA), transfer RNA (tRNA), non-coding RNA (ncRNA), and circular RNA (circRNA)) and DNA (such as genomic DNA (gDNA), cell free DNA (cfDNA), circulating tumor DNA (ctDNA)) and their fragments which can be useful in diagnosis, prognosis and monitoring of numerous diseases. Thus, exosomes and microvesicles can provide biomarkers for diseases (for example, including, but not limited to, the isolation of vesicles from urine for the assessment of renal disease). Target compounds that can be extracted using the devices and methods herein disclosed include proteins, lipids, antibodies, vitamins, minerals, steroids, hormones, cholesterol, amino acids, vesicles, exosomes, and nucleic acids.

In several embodiments, the samples described herein are biological fluid samples. In some embodiments, biological fluid samples are processed. As used herein, a “bodily fluid” shall be given its ordinary meaning and shall also refer to a sample of fluid collected from the body of the subject, including but not limited to, for example, blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the sample may be obtained from human, dog, pig, mouse, mammal, etc.

In one aspect, the method of isolating nucleic acids described herein comprises passing at least a part of a first solution comprising the sample through a capture material. The sample comprises target vesicles. In some embodiments, a sample obtained from a subject may be applied to the capture material without any dilution of the sample. In additional embodiments, a sample obtained from a subject may be diluted in a solution prior to being applied to the capture material.

In several embodiments, vesicle and nucleic acid capture material (“capture material”) is made from any suitable material that can retain the target vesicles being extracted from a sample and target nucleic acids encapsulated in or associated with the vesicles. In several embodiments, the material used for capture material is optimized to balance the attractive nature of the material for the target component and the ability of the material to release the target component under appropriate conditions.

In some embodiments, capture material is optionally modified to tailor the profile of target components retained by capture material. In some embodiments, capture material is electrocharged (e.g., electrostatically charged), coated with hydrophilic or hydrophobic materials, chemically modified, and/or biologically modified. In several embodiments, the zeta potential of capture material is used as a basis for modification (e.g., electrostatic charging) of the material. In some embodiments, capture material (based on its zeta potential) does not require modification. In some embodiments, capture material is modified by attaching a nucleotide sequence to the surface of capture material. In some embodiments, a protein is attached to the surface of capture material. In some embodiments, biotin or streptavidin is attached to the surface of capture material. In some embodiments, an antibody or antibody fragment is attached to capture material. Any of such embodiments can be employed to advantageously increase the efficiency of capture of a target.

In some embodiments, the interactions between vesicles and capture material and between vesicle associated nucleic acids and capture material are based on electrostatic interaction, hydrophobic interaction, van der Waals force, or a combination of these interactions. Thus, the biochemical makeup of the sample comprising the vesicles can alter these forces, possibly to a degree that significantly hampers the capture efficiency.

In some embodiments, a capture device comprises of a container to hold liquid samples and a capture material to capture the vesicles and vesicle associated nucleic acids through the capture material. In several embodiments, the capture material has a porous structure such as filter, beads and fiber to filter through the liquid samples through the capture material. A capture material can be positioned at the bottom of the container, therefore liquid samples can be placed in the container followed by filtration through the capture material at the bottom by way of application of positive pressure, negative pressure, centrifugal force, vacuum or gravitational flow. In order to process multiple liquid samples simultaneously with standard molecular biology techniques, a capture device has multiple containers which a capture material is positioned at the bottom of each container, for example, 8-well, 12-well, 24-well, 96-well, 384-well and 1536-well microplate format filterplates.

In several embodiments, the capture material herein comprises a single layer of filter material. In several embodiments, capture material comprises a plurality of layers of filter materials. In several embodiments, capture material comprises at least a first layer and a second layer of filter materials, in which the first layer is on top of the second layer or placed on the upstream surface of the second layer. In some embodiments, a sample is passed through the first layer of filter material to capture components that are about 0.5 μm, about 0.6 μm, about 0.7 μm, about 0.8 μm, about 0.9 μm, about 1.0 μm, about 2.0 μm, about 3.0 μm or greater in diameter in fluid samples. By removing the large components in the first layer of filter material, rapid filtration of liquid samples is achieved and clogging of the filter materials is avoided. In several embodiments, the particle retention rate of the first layer of filter material is from about 0.8 μm to about 3.0 μm at particle retention efficiency of 98%. In some embodiments, the particle retention rate of the first layer of filter material is from about 0.5, 0.6, 0.7, 0.8, 0.9, 1.0, 2.0 or 3.0 μm to about 1.0, 1.5, 2.0, 2.5, 3.0 or 3.5 μm at particle retention efficiency of 98%. In several embodiments, the particle retention rate of the second layer of filter material is from about 0.6 μm to about 1.2 μm at particle retention efficiency of 98%. In some embodiments, the particle retention rate of the second layer of filter material is smaller than or the same as the particle retention rate of the first layer of filter material. In some embodiments, the particle retention rate of the second layer of filter material is from about 0.5, 0.6, 0.7, 0.8, 0.9, 1.0, 1.1, or 1.2 μm to about 0.9, 1.0, 1.1, 1.2 or 1.3 μm at particle retention efficiency of 98%. In some embodiments, a sample is passed through the capture material in the first and/or second layers described herein so as to capture vesicles (e.g., exosomes, microvesicles, and other vesicles) having a size range from about 1 nm to about 1000 nm, from about 2 nm to about 500 nm, from about 3 nm to about 300 nm, from about 4 nm to about 200 nm, from about 5 nm to about 100 nm, in diameter. In additional embodiments, a sample is passed through the filter material in the first and/or second layers described herein so as to capture vesicles (e.g., exosomes, microvesicles, and other vesicles) having a diameter from about 1, 10, 20, 30, 40, 50, 60, 70, 80, 90, 100, or 110 nm to about 100, 200, 300, 400, 500, 600, 700, 800, 900, 1000, 1100, 1200, 1300, 1400, 1500, 2000, or 3000 nm.

In several embodiments, capture material comprises glass-like material or combinations of glass-like and non-glass-like materials. In some embodiments, the capture material comprises glass-like materials, which have a structure that is disordered, or “amorphous” at the atomic scale. The capture material may comprise a configuration including, but not limited to, sheet, filter, bead, fiber, coating, or other configurations. The capture material herein may comprise a material selected from the group consisting of silicon dioxide, metal oxide, mixed metal oxide, aluminum oxide, hafnium oxide, zirconium oxide, and combinations thereof. The capture material may include, but are not limited to, a material selected from the group consisting of nitrocellulose, nylon, polyvinylidene fluoride (PVDF), other similar polymers, nano-metal fibers, polystyrene, ethylene vinyl acetate, other co-polymers, natural fibers (e.g., silk), alginate fiber, and combinations thereof.

In some embodiments, vesicles are retained on or in capture material by virtue of the vesicle having physical dimensions that prohibit the vesicle from passing through the spaces of capture material (e.g., physical retention based on size). In some embodiments, vesicles are retained by the capture material by bonding forces between the vesicle and capture material. In some embodiments, vesicles form antigen-antibody bonds with the capture material. In several embodiments, vesicles form hydrogen bonds with capture material. In some embodiments, van der Waals forces form between the vesicle and capture material. In some embodiments, nucleotide sequences of the vesicle bind to nucleotide sequences attached to the capture material.

In some embodiments, differential capture of vesicles is achieved based on the surface expression of protein markers and a complementary agent on vesicle capture material which identifies that marker (e.g., an antibody that recognizes an antigen on a particular vesicle). In some embodiments, the markers are unique vesicle proteins or peptides. In some disease states, the markers may also comprise certain vesicle modifications, which, in some embodiments, are used to isolate particular vesicles. In such embodiments, vesicle capture material may be configured in a manner which allows for specific recognition of the vesicle modification. Modification of the vesicles may include, but are not limited to the addition of lipids, carbohydrates, and other molecules, such as acylated, formylated, lipoylated, myristolylated, palmitoylated, alkylated, methylated, isoprenylated, prenylated, amidated, glycosylated, hydroxylated, iodinated, adenylated, phosphorylated, sulfated, selenoylated, and ubiquitinated. In some embodiments, vesicle capture material is configured to recognize vesicle markers comprising non-proteins, such as lipids, carbohydrates, nucleic acids, RNA, mRNA, siRNA, microRNA, DNA, etc.

In several embodiments, a target range for capture conditions that the vesicles are exposed to when passed over/through the capture materials comprise between about 1 mM and about 5000 mM monovalent cation (e.g., sodium and/or potassium), including ranges having a lower concentration of about 10 mM, about 50 mM, about 100 mM, about 200 mM, about 300 mM, about 400 mM, about 500 mM, about 600 mM, about 700 mM, about 800 mM, about 900 mM, or about 1000 mM (and any concentration therebetween) and upper concentrations of about 1000 mM, about 2000 mM, about 3000 mM, about 4000 mM, or about 5000 mM (and any concentration therebetween). Thus, in several embodiments the concentration ranges are from about 300 mM to about 4000 mM, 400 mM to about 3000 mM, 500 mM to about 2000 mM, 600 mM to about 1000 mM, and overlapping ranges thereof. In conjunction with those conditions, the pH is adjusted, in several embodiments, from about 4, about 5, or about 6 to about 9 or about 10 (or pH values between those listed). Thus, depending on the embodiment, pH ranges include from about 4 to about 10, from about 5 to about 9, and from about 6 to about 9.

In several embodiments, it is advantageous to adjust the biochemical characteristics of biological samples to the above preferred ranges (e.g., salt concentration, pH, etc.) prior to applying a sample to a capture material as described herein. In several embodiments, a buffer solution, such as phosphate buffer saline (PBS) S) or HEPES buffer, may be added to the sample. In several embodiments, the pH of such buffers ranges from a pH of about 6, 7 or 8 to about 7, 8 or 9. In several embodiments, the concentration of monovalent cations, such as sodium and potassium, in the buffer is greater than about 100 mM, greater than about 500 mM, greater than about 1000 mM, greater than about 2000 mM, greater than about 3000 mM, and sometimes may require even greater concentrations, depending on the embodiment. In several embodiments, the final solution to be applied to a capture material (i.e., the mixture of the urine and buffer solution) has between about 600 mM to about 1000 mM monovalent cation, such as sodium and potassium, and from about pH 4, 5, 6, 7, or 8 to about pH 5, 6, 7, 8, 9 or 10.

In one aspect, the method described herein comprises adding a second solution containing a chaotropic reagent and alcohol to the capture material. The second solution may be a lysis and/or binding (“lysis/binding”) solution. Once vesicles are captured by capture material, applying a lysis/binding solution to the capture material may break the membranes of the vesicles and release vesicles associated nucleic acids from the vesicles. Spontaneously, the released nucleic acids may bind to the capture material due to the interaction forces between nucleic acids and capture material. In some embodiments, the capture material described herein may be incubated with lysis/binding solution for a duration from about 0, 10, 20, or 30 minutes to 10, 20, 30 or 40 minutes at a temperature from about 1, 2, 3, 4, 5, 6, 7, 8, 9, or 10° C. to 20, 25, 30, 35, or 40° C. In some embodiments, ideal configurations of capture material may be filter, mesh, fiber, porous structure, or any other surfaces with a high surface-to-volume ratio so as to avoid releasing the nucleic acids to solution phase instead of binding to the capture material. After the binding of nucleic acids by the capture material, the capture material is removed from the lysis/binding solution. For example, a lysis/binding solution is passed through the capture material by way of application of positive pressure, negative pressure, centrifugal force, vacuum or gravitational flow.

In some embodiments, the lysis/binding solution suitable for the efficient capture of nucleic acid by the capture material may contain (i) a chaotropic reagent, such as guanidinium isothiocyanate (GITC) and urea, and (ii) alcohol, such as ethanol and isopropyl alcohol. A concentration of GITC may be from about 1.0, 2.0, 3.0, 4.0, 4.5, 5.0 or 5.5 to about 3.0, 4.0, 5.5, 6.0 or 6.5M, from about 5.0M to 6.0M, or from 0.4M to 4M in the lysis/binding solution. A concentration of alcohol (e.g., ethanol) may be from about 10, 20, 30, 40, 50, 60, 70, 80% to 20, 30, 40, 50, 60, 70, 80, 90 or 99% in the lysis/binding solution. A concentration of alcohol (e.g., ethanol) for isolation of long nucleic acids having longer than 25 nucleotides, may be from about 20% to about 80%, or from about 40% to about 70%. A content of alcohol (e.g., ethanol) for isolation of short nucleic acids having 25 nucleotides long or shorter, may be from 50% to 99.9%, most preferably from 70% to 95%. A pH range of a lysis/binding solution may be from pH5 to pH9.4, from pH 6 to pH9, pH7 to pH8.6.

In one aspect, the method described herein may comprise rinsing the capture material described above with a third solution. After capturing the vesicle associated nucleic acids by the capture material, the capture material can be rinsed at least once with a wash solution to remove vesicle debris, non-nucleic-acid vesicle components and lysis/binding solution components. Wash solution suitable for this method may contain alcohol, such as ethanol, and optionally includes chaotropic reagent, such as guanidinium isothiocyanate (GITC). A concentration of alcohol (e.g, ethanol) in the wash solution may be from 30% to 99.9%, more preferably from 40% to 95%. In some embodiments, a concentration of alcohol in the wash solution may be from about 20, 30, 40, 50, 60, 70, 80, 90, 95 or 98% to about 30, 40, 50, 60, 70, 80, 90, 95, or 99.9%. A concentration of GITC may be from 0M to 2M. A concentration of GITC may be from about 0, 0.5, 1.0, 1.5, 2.0 M to about 0.5, 1.0, 1.5, 2.0, 2.5 M. In some embodiments, a concentration of GITC may be lower than or same as the GITC concentration of the lysis/binding solution described herein. More than one wash solution could be used to further remove contaminants, for example, the first wash solution contains both alcohol and chaotropic reagent to remove biological contaminants efficiently, and the second wash solution contains alcohol only to remove chaotropic reagent from lysis/binding solution and first wash solution. For each wash step, a wash solution may be passed through the capture material by way of application of positive pressure, negative pressure, centrifugal force, vacuum or gravitational flow. Optionally, the capture material is dried at 0° C. to 40° C. for 0 to 30 min at ambient or vacuum condition to evaporate residual alcohol.

In one aspect, the method described herein may comprise passing a fourth solution through the capture material described above to elute RNA from the capture material to the fourth solution. Nucleic acids captured by the capture material may be collected in a small volume of an elution solution. Elution solution suitable for this method may be a nuclease-free water or buffer with less than 100 mM salt and less than 20% alcohol. The elution solution may be a butter with less than about 100, 90, 80, 70, 60, 50, 40, 30, or 20 mM salt, and/or less than about 30, 20, 10, 9, 8, 7, 6, 5, or 1% alcohol. The elution solution containing low concentration of a mild detergent, such as 0.01% to 0.5% Tween20, may be useful to wet the capture material spontaneously to allow rapid release of vesicle associated nucleic acids into the elution solution. Optionally, the capture material can be incubated with an elution solution at a temperature from about 0, 10, 20, 30, 37 or 40° C. to about 10, 20, 30, 37 or 40° C. for a duration from about 0, 10, 20, or 30 minutes to about 10, 20 or 30 minutes. Nucleic acid in elution solution may be collected by being passed through the capture material by way of application of positive pressure, negative pressure, centrifugal force, vacuum or gravitational flow. In some embodiments, the RNA eluted from the capture material comprises at least one RNA selected from the group consisting of mRNA, microRNA (miRNA), circular RNA (circRNA), and long non-coding RNA (lncRNA). In some embodiments, the RNA comprises at least one RNA selected from the group consisting of microRNA (miRNA), circular RNA (circRNA), and long non-coding RNA (lncRNA).

In one aspect, the method described herein is performed in less than 4, 3, 2, or 1 hour. In one aspect, the method described herein is performed in less than 3 hour. In one aspect, the method described herein is performed in less than 2 hour. In another aspect, the method uses centrifugation from about 200, 300, 400, 500, 600, 700, 800, 900, 1000, 2000, 3000, 4000 or 5000×g to about 1000, 2000, 3000, 4000, 5,000, 6000, 7000 or 8000×g. In another aspect, the method uses centrifugation from 500×g to 5,000×g, 100×g to 4000×g, 1000×g to 3000×g, or 800×g to 3500×g.

### EXAMPLES

### Example 1: Urine Extracellular Vesicle mRNA Assay

Spot urine was obtained from a healthy volunteer. Cells and large particles were removed by centrifugation at 800×g for 15 min. Urine supernatant was mixed with ¼ volume of 25× Phosphate Buffered Saline (PBS), pH 7.4, and 12.5 mL each of the mix was filtered with glass fiber filter tube with various glass fiber filter materials A to F or their combinations (Table 1) by centrifugation at 3500×g for 2 min. Lysis/Binding solution were prepared by mixing equal volumes of 4M guanidine isothiocyanate (GITC), 50 mM Tris-HCl, pH 7.5, 25 mM EDTA solution and 100% Ethanol solution. Five hundred μL of each Lysis/Binding solution spiked with 1×10⁵ copies of synthetic Fluc mRNA (Integrated DNA technologies (IDT), IA) was added to the filter and incubated at 37° C. for 10 min, then filtered by centrifugation at 3500×g for 2 min. The filter was rinsed once with 1 mL of each Lysis/Binding solution and twice with 2.5 mL of 70% Ethanol. The filter was dried under vacuum for 5 min at room temperature under vacuum for 5 min and 80 μL of RNase/DNase free water was added. By centrifugation at 3500×g for 2 min, vesicle RNA was obtained in the filtrate.

mRNA was quantified by real-time reverse transcriptase polymerase chain reaction (RT-PCR). Four μL of RNA was used in twenty μL reverse transcription reaction containing 125 each of dNTPs, 10 μM random hexamer, 2.6 U/μL MMLV reverse transcriptase (Promega, WI) and 0.13 U/μL Ribonuclease inhibitor (Promega, WI) with the following temperature protocol: 25° C. for 5 min, 37° C. for 60 min and 85° C. for 5 min. Two μL of cDNA was used in five real-time PCR reaction using SsoAdvanced Universal SYBR Green Supermix (Bio-rad, CA) and 500 nM sense and antisense primers with the following temperature protocol: 95° C. for 10 min, 40 cycles of 95° C. for 30 sec and 65° C. for 60 sec, followed by melting curve analysis. Four genes (ACTB, GAPDH, ALDOB, Fluc) were quantitated with the primer pairs listed in Table 2.

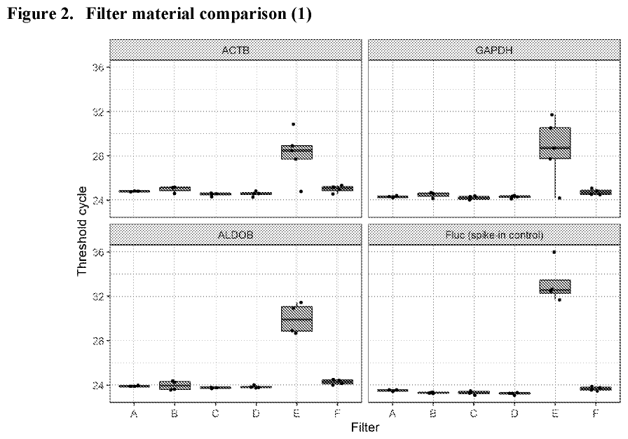

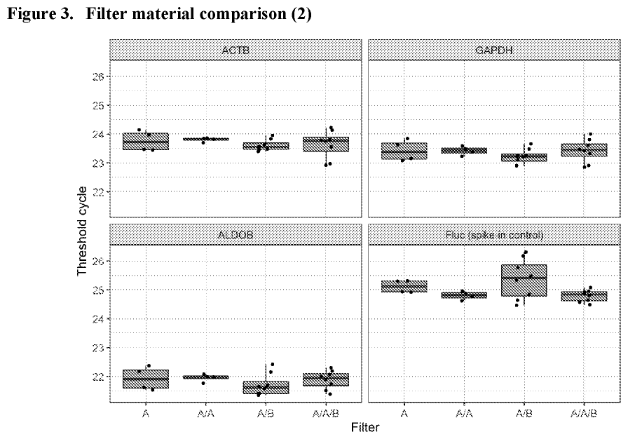

All the four genes were successfully detected and threshold cycle values of these genes became lowest or maximum RNA yield was obtained with glass fiber filters A, B, C, D, F and combinations of A and B (FIG. 2, FIG. 3).

### Example 2: Urine Extracellular Vesicle mRNA Assay

Spot urine was obtained from a healthy volunteer. Cells and large particles were removed by centrifugation at 800×g for 15 min. Urine supernatant was mixed with ¼ volume of 25×PBS, pH 7.4, and 600 μL each of the mix was filtered with 8-well filter strip with glass fiber filter combination A/A/B (Table 1) by centrifugation at 3500×g for 2 min. Several types of Lysis/Binding solution were prepared by mixing 4M GITC, 50 mM Tris-HCl, pH 7.5, 25 mM EDTA solution and 100% Ethanol solution at various ratios such as 4M GITC/0% Ethanol, 3.6M GITC/10% Ethanol, 3.2M GITC/20% Ethanol, 2.8M GITC/30% Ethanol, 2.4M GITC/40% Ethanol, 2M GITC/50% Ethanol, 1.6M GITC/60% Ethanol, 1.2M GITC/70% Ethanol, 0.8M GITC/80% Ethanol, 0.4M GITC/90% Ethanol, and 0M GITC/100% Ethanol. Sixty μL of each Lysis/Binding solution was added to the filter and incubated at 37° C. for 10 min, then filtered by centrifugation at 3500×g for 2 min. The filter was rinsed once with 300 μL of each Lysis/Binding solution and twice with 300 μL of 70% Ethanol. The filter was dried under vacuum for 5 min at room temperature and 60 μL of RNase/DNase free water was added. By centrifugation at 3500×g for 2 min, vesicle RNA was obtained in the filtrate.

mRNA was quantified by RT-PCR. Eight μL of RNA was used in twenty μL reverse transcription reaction using qScript XLT cDNA SuperMix (Quantabio, MA) with the following temperature protocol: 25° C. for 5 min, 42° C. for 60 min and 85° C. for 5 min. Two μL of cDNA was used in five μL real-time PCR reaction using SsoAdvanced Universal SYBR Green Supermix (Bio-rad, CA) and 500 nM sense and antisense primers with the following temperature protocol: 95° C. for 10 min, 40 cycles of 95° C. for 30 sec and 65° C. for 60 sec, followed by melting curve analysis. Four genes (ACTB, GAPDH, ALDOB, RPLP0) were quantitated with the primer pairs listed in Table 2.

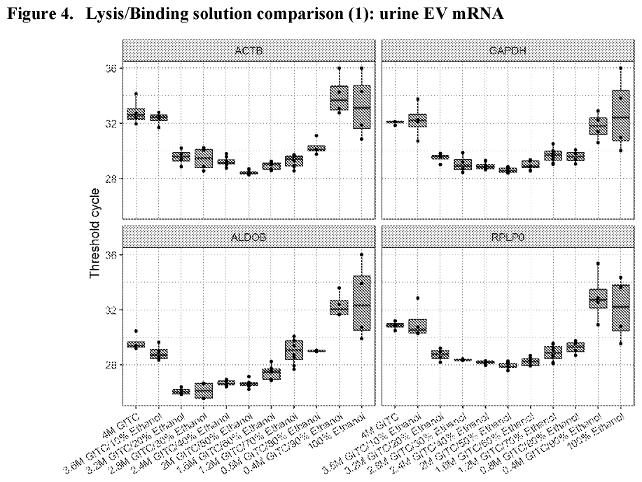

All the four genes were successfully detected and threshold cycle values of these genes became lowest or maximum RNA yield was obtained with Lysis/Binding solution containing 0.8M to 3.2M GITC/20% to 70% Ethanol (FIG. 4).

### Example 3: Plasma Extracellular Vesicle mRNA Assay

EDTA plasma was obtained from a healthy volunteer. Cells and large particles were removed by centrifugation at 3500×g for 15 min. Supernatant was mixed with ¼ volume of 25×PBS, pH 7.4, and 200 μL each of the mix was filtered with 8-well filter strip with glass fiber filter combination A/A/B (Table 1) by centrifugation at 3500×g for 2 min. Several types of Lysis/Binding solution were prepared by mixing 4M GITC, 50 mM Tris-HCl, pH 7.5, 25 mM EDTA solution and 100% Ethanol solution at various ratios such as 4M GITC/0% Ethanol, 3.6M GITC/10% Ethanol, 3.2M GITC/20% Ethanol, 2.8M GITC/30% Ethanol, 2.4M GITC/40% Ethanol, 2M GITC/50% Ethanol, 1.6M GITC/60% Ethanol, 1.2M GITC/70% Ethanol, 0.8M GITC/80% Ethanol, 0.4M GITC/90% Ethanol, and 0M GITC/100% Ethanol. One hundred μL of each Lysis/Binding solution was added to the filter and incubated at 37° C. for 10 min, then filtered by centrifugation at 3500×g for 2 min. The filter was rinsed once with 500 μL of each Lysis/Binding solution and twice with 300 μL of 70% Ethanol. The filter was dried under vacuum for 5 min at room temperature and 60 μL of RNase/DNase free water was added. By centrifugation at 3500×g for 2 min, vesicle RNA was obtained in the filtrate.

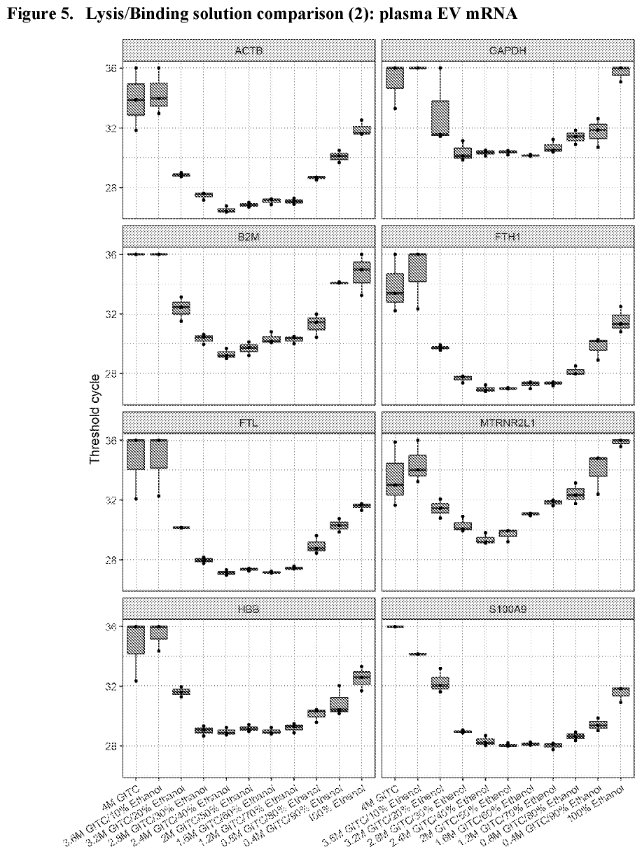

mRNA was quantified by real-time RT-PCR as described in Example 2. Eight genes (ACTB, GAPDH, B2M, FTH1, FTL, MTRNR2L1, HBB, S100A9) were quantitated with the primer pairs listed in Table 2. All the eight genes were successfully detected and threshold cycle values of these genes became lowest or maximum RNA yield was obtained with Lysis/Binding solution containing 1.2M to 2.8M GITC/30% to 70% Ethanol (FIG. 5).

### Example 4: Urine Extracellular Vesicle mRNA Assay

Spot urine was obtained from a healthy volunteer. Cells and large particles were removed by centrifugation at 800×g for 15 min. Urine supernatant was mixed with ¼ volume of 25×PBS, pH 7.4, and 600 μL each of the mix was filtered with 8-well filter strip with glass fiber filter combination A/A/B (Table 1) by centrifugation at 3500×g for 2 min. Several types of Lysis/Binding solution were prepared by mixing equal volumes of 4M GITC, 50 mM Tris-HCl, pH 7.5, 25 mM EDTA solution and 100% Ethanol solution and adjusted pH4, pH5, pH6, pH6.5, pH7.5, pH8.5 and pH 9.5. Sixty μL of each Lysis/Binding solution was added to the filter and incubated at 37° C. for 10 min, then filtered by centrifugation at 3500×g for 2 min. The filter was rinsed once with 300 μL of each Lysis/Binding solution and twice with 300 μL of 70% Ethanol. The filter was dried under vacuum for 5 min at room temperature and 60 μL of RNase/DNase free water was added. By centrifugation at 3500×g for 2 min, vesicle RNA was obtained in the filtrate.

mRNA was quantified by real-time RT-PCR as described in Example 2. Four genes (ACTB, GAPDH, ALDOB, RPLP0) were quantitated with the primer pairs listed in Table 2.

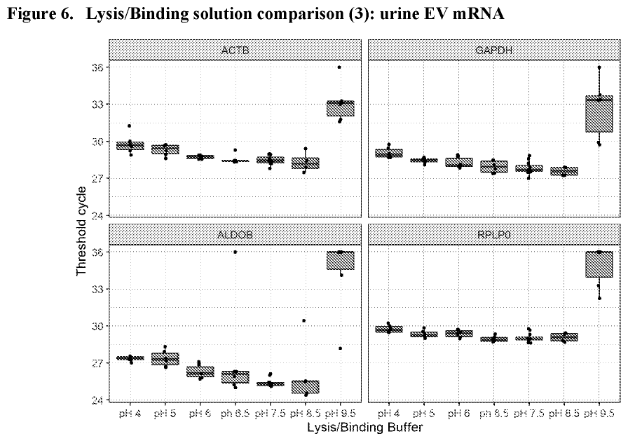

All the four genes were successfully detected and threshold cycle values of these genes became lowest or maximum RNA yield was obtained with Lysis/Binding solution pH 6 to pH8.5 (FIG. 6).

### Example 5: Extracellular Vesicle miRNA Assay

Extracellular vesicle RNA was isolated from urine or EDTA plasma following the procedures described in Examples 2 and 3. miRNA was quantified using polyadenylation/RT-qPCR method.

Four μL of RNA was used in ten μL miRNA reverse transcription reaction containing 25.6 U MMLV Reverse Transcriptase (Promega, WI), 1.28 U RNasin Ribonuclease Inhibitor (Promega), 1 U E. coli Poly(A) Polymerase (New England Biolabs, MA), 1× E. coli Poly(A) Polymerase Reaction Buffer, 1 mM ATP, 1 mM dNTP, 0.1 CAGGTCCAGTTTTTTTTTTTTTTTVN (V: A, G, or C, N: A, G, T or C) with the following temperature protocol: 5 min, 37° C. for 60 min and 85° C. for 5 min. One μL of cDNA was used in five μL real-time PCR reaction using SsoAdvanced Universal SYBR Green Supermix (Bio-rad, CA) and 100 nM sense and antisense primers with the following temperature protocol: 95° C. for 10 min, 40 cycles of 95° C. for 30 sec and 60° C. for 60 sec, followed by melting curve analysis.

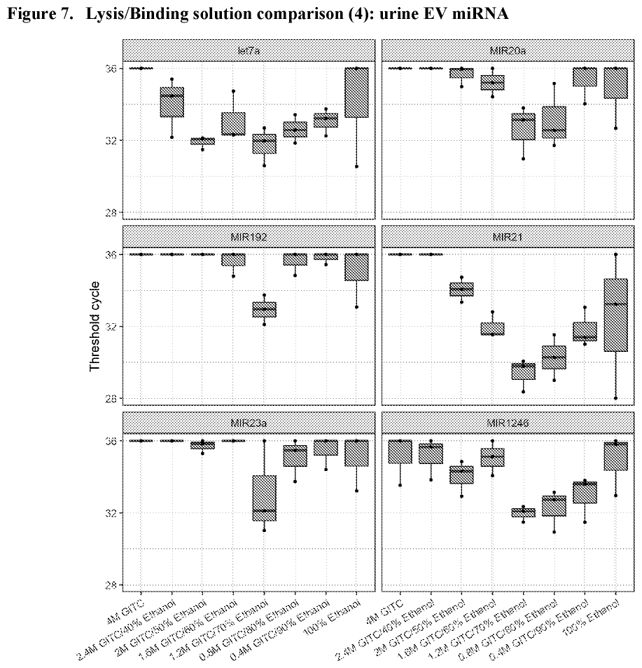

For urine EV RNA, six genes (let7a, miR20a, miR192, miR21, miR23a, miR1246) were quantitated with the primer pairs listed in Table 2. All the genes were successfully detected. Threshold cycle values of these genes became lowest or maximum RNA yield was obtained with Lysis/Binding solution containing 70% to 90% Ethanol (FIG. 7).

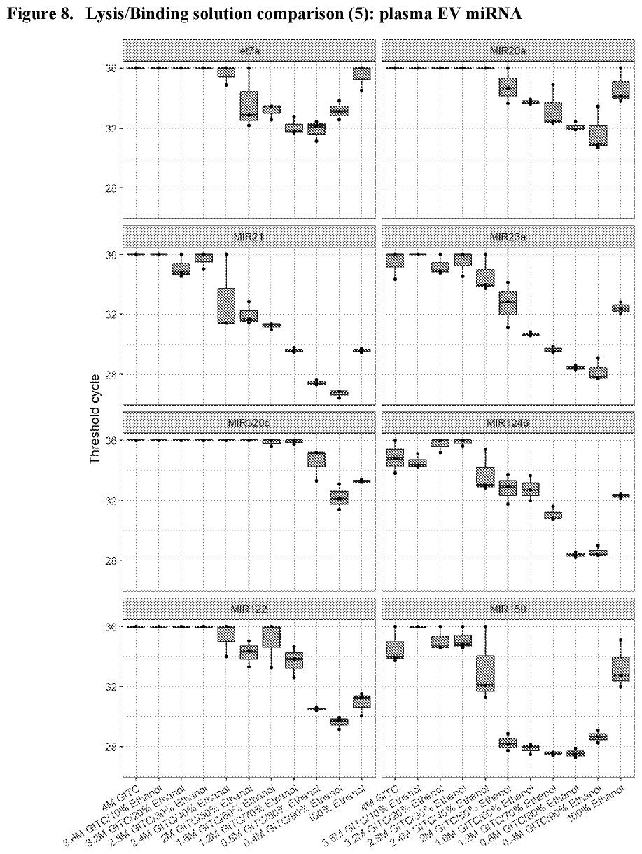

For plasma EV RNA, eight genes (let7a, miR20a, miR21, miR23a, miR320c, miR1246, miR122, miR150) were quantitated with the primer pairs listed in Table 2. All the genes were successfully detected. Threshold cycle values of these genes became lowest or maximum RNA yield was obtained with Lysis/Binding solution containing 70% to 90% Ethanol (FIG. 8).

### Example 6: Extracellular Vesicle mRNA Assay

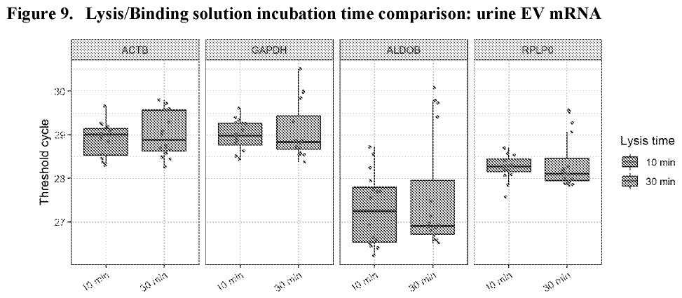

Extracellular vesicle RNA was isolated from urine following the procedures described in Example 2 except using extended 30-min incubation at 37° C. during vesicle lysis/RNA binding on the filter. Four genes (ACTB, GAPDH, ALDOB, RPLP0) were assayed comparing to the method described in Example 2 with 10 min incubation. No statistical significance was observed between 10-min and 30-min incubation, indicating that 10 min incubation was sufficient for vesicle lysis and RNA binding (FIG. 9).

### Example 7: Urine Extracellular Vesicle mRNA Assay

Spot urine was obtained from a healthy volunteer. Cells and large particles were removed by centrifugation at 800×g for 15 min. Ten mL urine supernatant was mixed with ¼ volume of 25×PBS, pH 7.4, and the mix was filtered with filter tube with glass fiber filter combination A/B (Table 1) at 3500×g for 5 min. Five hundred μL of Lysis/Binding solution (2M GITC, 25 mM Tris-HCl, pH 7.5, 12.5 mM EDTA, 50% Ethanol) was added to the filter and incubated at 37° C. for 10 min, then filtered by centrifugation at 3500×g for 2 min. The filter was rinsed once with 1 mL of Lysis/Binding solution and twice with 2.5 mL of various Wash solution containing 30% to 70% Ethanol. The filter was dried under vacuum for 5 min at room temperature and 80 μL of RNase/DNase free water was added. By centrifugation at 3500×g for 2 min, vesicle RNA was obtained in the filtrate.

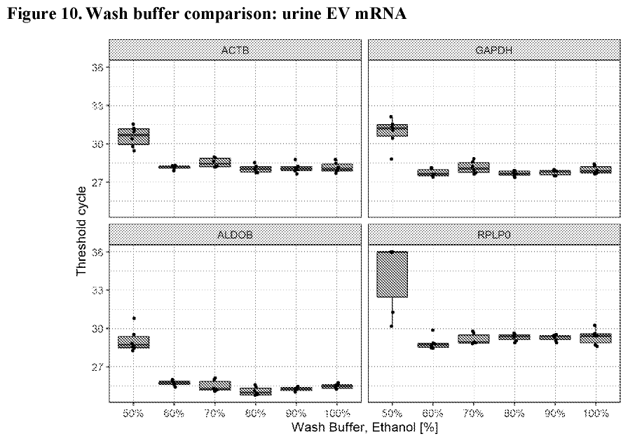

mRNA was quantified by real-time RT-PCR as described in Example 2. Four genes (ACTB, GAPDH, UMOD, RPLP0) were quantitated with the primer pairs listed in Table 2. All the four genes were successfully detected using various Wash buffer. Threshold cycle values of these genes became lowest or maximum RNA yield was obtained with Wash buffer containing 70% Ethanol (FIG. 10).

### Example 8: Urine Extracellular Vesicle mRNA Assay

EV RNA was captured by a glass fiber filter tube as described in Example 7 from 10 mL urine supernatant from a healthy donor. After the filter was rinsed and dried, 40 to 200 μL of RNase/DNase free water was added to the filter. By centrifugation at 3500×g for 2 min, vesicle RNA was obtained in the filtrate. The elution step was repeated twice.

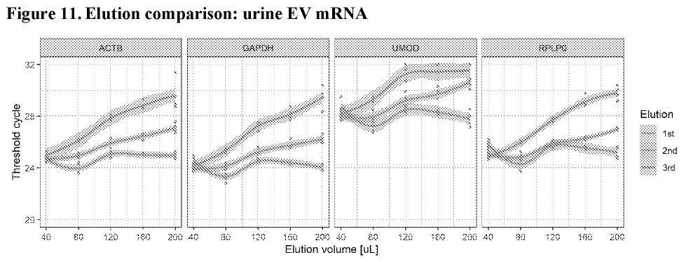

mRNA was quantified by real-time RT-PCR as described in Example 2. Four genes (ACTB, GAPDH, UMOD, RPLP0) were quantitated with the primer pairs listed in Table 2. All the four genes were successfully detected. Threshold cycle values of these genes became lowest or maximum RNA yield was obtained with 80 μL water in the first elution (FIG. 11). In the second and third elutions, RNA quantities were lower than in the first elution, indicating RNA was eluted efficiently in the first elution (FIG. 11).

### Example 9: Urine Extracellular Vesicle mRNA Assay

Spot urine was obtained from a healthy volunteer. Cells and large particles were removed by centrifugation at 800×g for 15 min. Forty mL urine supernatant was mixed with ¼ volume of 25×PBS, pH 7.4, and the mix was filtered with glass fiber filter tube at 3500×g for 5 min. Five hundred μL of Lysis/Binding solution A (2M GITC, 25 mM Tris-HCl, pH 7.5, 12.5 mM EDTA, 50% Ethanol), or Lysis/Binding solution B (1.2M GITC, 15 mM Tris-HCl, pH 7.5, 7.5 mM EDTA, 70% Ethanol) was added to the filter and incubated at 37° C. for 10 min, then filtered by centrifugation at 3500×g for 2 min. The filter was rinsed once with 1 mL of Lysis/Binding solution A or B and twice with 2.5 mL of 70% Ethanol. The filter was dried under vacuum for 5 min at room temperature and 80 μL of RNase/DNase free water was added. By centrifugation at 3500×g for 2 min, vesicle RNA was obtained in the filtrate. For reference, EV RNA was isolated by hybridization to oligo(dT)-immobilized microplate or by RNeasy Mini Kit (Qiagen, MD) following the manufacturer's protocol.

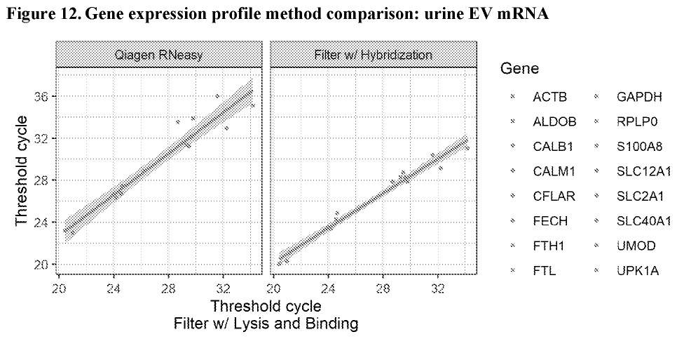

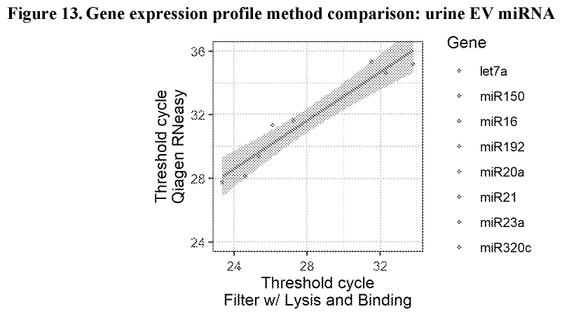

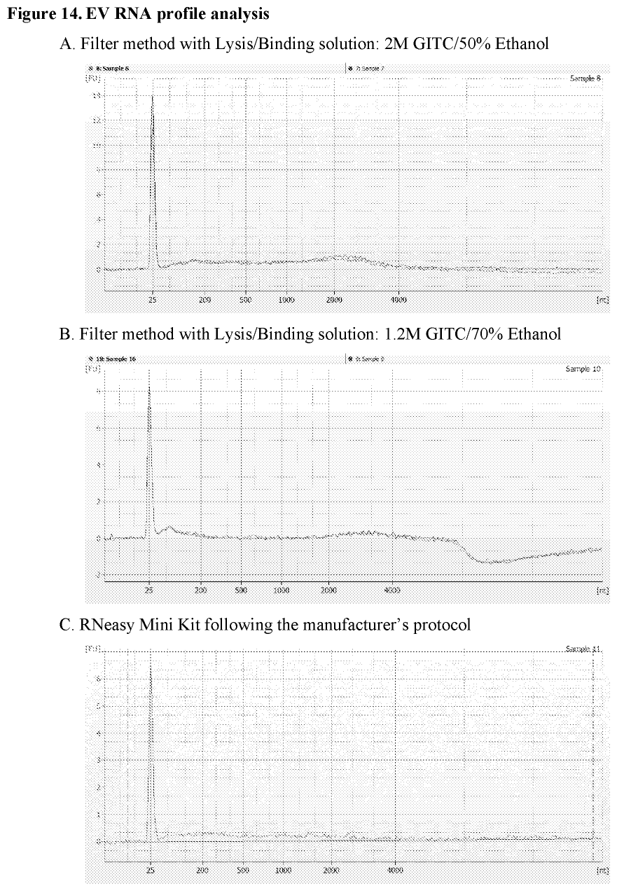

Sixteen mRNA and eight miRNA were quantified by real-time PCR as described in Example 2 and 4, respectively. Gene expression profiles of mRNA and miRNA obtained by the filter method described here showed high correlation with those obtained by the reference methods yet gave lower threshold cycles or higher RNA recovery yields than the reference methods (FIG. 12, FIG. 13, Table 3). RNA profiles by Bioanalyzer (Agilent, CA) and RiboGreen RNA assay (Life Technologies, CA) also indicated that RNA yield obtained using the filter method described here are more abundant compared to the reference methods (FIG. 14, Table 4).

### Tables and Figures

TABLE 1
Filter material parameters

| | Thickness | Weight | Particle retention | Filtration |
|---|---|---|---|---|
| Filter | [mm] | [g/m²] | rate* [μm] | Speed [mls/min] |
| A | 0.44 | 88 | 0.7 | 25 |
| B | 0.25 | 53 | 1 | 77.5 |
| C | 0.32 | 68 | 1.1 | 102 |
| D | 0.28 | 56 | 1.2 | 130 |
| E | 0.66 | 144 | 1 | 48 |
| F | 0.66 | 123 | 3.1 | 350 |

*Particles retention efficiency of 98%

TABLE 2
Primer sequences

| Species | Gene | Sense sequence | Antisense sequence |
|---|---|---|---|
| Human | ACTB | TTTTTCCTGGCACCCAGCACA | TTTTTGCCGATCCACACGGAGTACT |
| | | AT | |
| Human | GAPDH | CCCACTCCTCCACCTTTGAC | CATACCAGGAAATGAGCTTGACAA |
| Human | ALDOB | AACCACCATTCAAGGGCTTG | TTGGCGTTTTCCTGGATAGC |
| Human | RPLP0 | TGCATCAGTACCCCATTCTAT | GGTGTAATCCGTCTCCACAGACA |
| | | CA | |
| Human | B2M | TGACTTTGTCACAGCCCAAGA | AATGCGGCATCTTCAAACCT |
| | | TA | |
| Human | FTH1 | ACTGGAACTGCACAAACTGG | ATCTTGCGCAAGTTGGTCAC |
| Human | FTL | ATGAGCTCCCAGATTCGTCAG | AGGCCTGCAGGTACAAATTG |
| Human | MTRNR2L1 | TGTTTAATGGCTGCGGTACC | TTCACGGGCAGGTCAATTTC |
| Human | HBB | GCCCATCACTTTGGCAAAGA | CCAGCCACCACTTTCTGATAGG |
| Human | S100A9 | CTGAGCTTCGAGGAGTTCATC | CGTCACCCTCGTGCATCTT |
| | | A | |
| Human | CALB1 | CGTATTACCCACAGAAGAGAA | CCATGTCTTCATGAATTCCTCACA |
| | | TTTCC | |
| Human | CALM1 | TGACAAGGATGGCAATGGTTA | TACTTCTTCATCTGTTAGTTTTTCTC |
| | | TA | CTAAGT |
| Human | CFLAR | CAATTTGCCTGTATGCCCGA | GCCCTCTGACACCACATAGT |
| Human | FECH | ACAGGCAGCAGCTTAAATGC | TCTGCAAAGCACTGGATGAG |
| Human | S100A8 | CAGGAGTTCCTCATTCTGGTG | TTGTGGCTTTCTTCATGGCTTT |
| | | AT | |
| Human | SLC12A1 | ACTCCAGAGCTGCTAATCTCA | AACTAGTAAGACAGGTGGGAGGTT |
| | | TTGT | CT |
| Human | SLC2A1 | TCATTGTGGGCATGTGCTTC | ACCAGGAGCACAGTGAAGATG |
| Human | SLC40A1 | ACAACCAGCCTGTGTTTCTG | TGTGGTGATGCAGTCAAAGC |
| Human | UMOD | CCTGAACTTGGGTCCCATCA | GCCCCAAGCTGCTAAAAGC |
| Human | UPK1A | ATCCCTGATCACCAAGCAGAT | AAGGCTGACGTGAAGTTCAC |
| | | G | |
| Human | let7a | GCAGTGAGGTAGTAGGTTGT | GGTCCAGTTTTTTTTTTTTTTTAACT |
| | | | ATAC |
| Human | MIR16 | CGCATAGCAGCACGTA | CCAGTTTTTTTTTTTTTTTCGCCA |
| Human | MIR20a | ACAGTAAAGTGCTTATAGTGC | GTCCAGTTTTTTTTTTTTTTTCTACCT |
| | | A | |
| Human | MIR21 | TCAGTAGCTTATCAGACTGAT | CGTCCAGTTTTTTTTTTTTTTTCAAC |
| | | G | |
| Human | MIR23a | CATCACATTGCCAGGGAT | CGTCCAGTTTTTTTTTTTTTTTGGAA |
| Human | MIR122 | ACAGTGGAGTGTGACAATG | TCCAGTTTTTTTTTTTTTTTCAAACA |
| | | | C |
| Human | MIR150 | GTCTCCCAACCCTTGTAC | GTCCAGTTTTTTTTTTTTTTTCACTG |
| Human | MIR192 | CGCACTGACCTATGAATTGA | TCCAGTTTTTTTTTTTTTTTGGCTG |
| Human | MIR320c | CGCAAAAGCTGGGTTGA | AGGTCCAGTTTTTTTTTTTTTTTACC |
| | | | CTC |
| Human | MIR939 | CTGGGGAGCTGAGGCTC | AGGTCCAGTTTTTTTTTTTTTTTCAC |
| | | | CCC |
| Human | MIR939 | CTGGGGAGCTGAGGCTC | CAGGTCCAGTTTTTTTTTTTTTTTCA |
| | | | CC |
| Human | MIR1202 | CGTGCCAGCTGCA | CAGGTCCAGTTTTTTTTTTTTTTTCT |
| | | | CC |
| Human | MIR1246 | CGCAGAATGGATTTTTGGAG | CAGGTCCAGTTTTTTTTTTTTTTTCC |
| | | | TGCT |
| Human | MIR1290 | CGCAGTGGATTTTTGGATC | CAGGTCCAGTTTTTTTTTTTTTTTCC |
| | | | CTGA |

TABLE 3
EV RNA yield comparison (1)

| | EV RNA recovery [%] | | | | | | |
|---|---|---|---|---|---|---|---|
| Method | ACTB | ALDOB | FTH1 | FTL | GAPDH | RPLP0 | mean |
| Filter (2M GITC/50% | 29.9 | 15.6 | 17.2 | 28.6 | 24.0 | 15.5 | 21.8 |
| Ethanol) | | | | | | | |
| RNeasy Mini Kit | 9.7 | 13.6 | 7.5 | 11.5 | 8.3 | 6.1 | 9.4 |

TABLE 4
EV RNA yield comparison (2)

| Method | Lysis/Binding solution | RNA [ng] |
|---|---|---|
| Filter method | 2M GITC/50% | 13.5 |
| | Ethanol | |
| Filter method | 1.2M GITC/70% | 7.74 |
| | Ethanol | |
| RNeasy Mini | — | 8.14 |
| Kit | | |

1. A method of isolating nucleic acids from vesicles in a sample, comprising:
passing at least a part of a first solution comprising the sample through a capture material, wherein the capture material includes a material selected from the group consisting of silicon dioxide, metal oxide, or combinations thereof, wherein vesicles from said sample are captured on or in the capture material;
adding a second solution containing a chaotropic reagent and alcohol to the capture material;
rinsing the capture material with a third solution containing alcohol; and
passing a fourth solution through the capture material to elute RNA from the capture material to the fourth solution.

2. The method according to claim 1, further including, prior to the passing,
adjusting a salt concentration of the sample to between 300 mM and 4000 mM, and/or
adjusting a pH of the sample to a pH from pH 4 to pH 10.

3. The method according to claim 1, wherein the chaotropic reagent comprises guanidinium isothiocyanate (GITC).

4. The method according to claim 3, wherein a concentration of GITC in the second solution is from 0M to 6M.

5. The method according to claim 3, wherein a concentration of GITC in the second solution is from 0.4M to 4M.

6. The method according to claim 1, wherein the RNA comprises RNA that is longer than 25 nucleotides, and the alcohol in the second solution comprises ethanol.

7. The method according to claim 6, wherein a concentration of ethanol in the second solution is from 20% to 80%.

8. The method according to claim 6, wherein a concentration of ethanol in the second solution is from 40% to 70%.

9. The method according to claim 1, wherein the RNA comprises RNA that is 25 nucleotides or shorter, and the alcohol in the second solution comprises ethanol at a concentration from 50% to 99.9% of the second solution.

10. The method according to claim 1, wherein the RNA comprises RNA that is 25 nucleotides or shorter, and the alcohol in the second solution comprises ethanol at a concentration from 70% to 95% of the second solution.

11. The method according to claim 1, wherein the alcohol in the third solution contains ethanol at a concentration from 30% to 99.9% of the third solution.

12. The method according to claim 1, wherein the alcohol in the third solution contains ethanol at a concentration from 40% to 95% of the third solution.

13. The method according to claim 1, wherein the fourth solution comprises
water, or
a buffer with less than 100 mM salt and less than 20% alcohol.

14. The method according to claim 1, wherein the fourth solution consists of
water, or
a buffer with less than 100 mM salt and less than 20% alcohol.

15. The method according to claim 1, wherein said sample is a urine, plasma or serum sample.

16. The method according to claim 1, wherein said sample is from human.

17. The method according to claim 1, wherein the RNA comprises at least one RNA selected from the group consisting of mRNA, microRNA (miRNA), circular RNA (circRNA), and long non-coding RNA (lncRNA).

18. The method according to claim 1, wherein the RNA comprises at least one RNA selected from the group consisting of microRNA (miRNA), circular RNA (circRNA), and long non-coding RNA (lncRNA).

19. The method according to claim 1, wherein the method is performed in less than 3 hours.

20. The method of claim 1, wherein the method uses centrifugation from 500×g to 5,000×g.

21. The method of claim 1, wherein the capture material includes filter, bead, fiber, or coating.

22. The method of claim 1, wherein the metal oxide includes aluminum oxide, hafnium oxide, or zirconium oxide.

23. The method of claim 1, wherein the vesicles include an exosome having a diameter from 50 to 100 nm.

24. The method of claim 1, wherein the capture material comprises at least two layers.

25. The method of claim 1, wherein
a top layer of said at least two layers has a particle retention rate from 0.8 μm to 1.3 μm at particle retention efficiency of 98%, and
a bottom layer of said at least two layers has a particle retention rate from about 0.6 μm to about 1.2 μm at particle retention efficiency of 98%.
